(19) United States
(12) Patent Application Publication
(10) Pub. No.: US 2016/0051353 A1
(43) Pub. Date: Feb. 25, 2016

(54) High-Throughput Organ-Targeted Microinjection System

(71) Applicant: Massachusetts Institute ofTechnology, Cambridge, MA (US)

(72) Inventors: Mehmet Fatih Yanik, Watertown, MA (US); Tsung-Yao Chang, Cambridge, MA (US); Peng Shi, Hong Kong (HK)

(21) Appl. No.: 14/821,905

(22) Filed: Aug. 10, 2015

Related U.S. Application Data
(60) Provisional application No. 62/039,597, filed on Aug. 20, 2014.

(51) Int. Cl. A61D 7/00; A61B 5/00; A61D 3/00; A61K 49/00

(52) U.S. Cl. CPC A61D 7/00; A61K 49/0008; A61B 5/0059; A61B 5/4848; A61D 3/00; A61B 5/0042; A61B 5/0044; A61B 2019/202

(57) ABSTRACT

Automatic system for efficient delivery of biologics into target organs of zebrafish larvae for high-throughput in vivo screening. The system includes a reservoir containing zebrafish larvae immersed in a hydrogel in its liquid state. A microfluidic component removes a droplet of the hydrogel having a single zebrafish larva contained therein and deposits the droplet on a surface for receiving an array of hydrogel droplets. Structure or substances is provided for inducing the larva to assume a dorsal or lateral orientation within the droplet. A cooler cools the surface to solidify the hydrogel droplets thereby to immobilize the larvae for observation by an optical arrangement that identifies target organs in each larva using an image template-matching algorithm. A pressure driven microinjection needle injects biologics into the target organ of the zebrafish larva for screening studies.

This application claims priority to provisional implication Ser. No. 62/039,597 filed Aug. 20, 2014, the contents of which are incorporated herein by reference.

### BACKGROUND OF THE INVENTION

This invention relates to high-throughput screening and more particularly to a system for injecting biologics into zebrafish larvae for in vivo screens.

Biologics such as nucleic acids^{1,2}, proteins³, cells⁴, and nanoparticle vehicles for drug delivery⁵ are currently under active investigation as therapeutics for a wide variety of human diseases. In contrast to chemically synthesized small molecules with enhanced solubility and permeability, these molecules have structures that are generally much larger and far more complex, and therefore require sophisticated modes of delivery⁶⁻⁹. Consequently, although large libraries of biologics and delivery vehicles are currently available¹⁰⁻¹³, it remains challenging to rapidly assess their in vivo properties such as delivery efficiency, biodistribution, pharmacokinetics, tissue specificity, efficacy, and toxicity.

Zebrafish (Danio rerio) are being increasingly used for large-scale in vivo chemical and genetic screens. A combination of features, including small size, optical transparency, and rapid organogenesis, make zebrafish a vertebrate model that is uniquely suited for high-throughput screening (HTS)¹⁴⁻¹⁶, which is cost-prohibitive in mammals. HTS of small molecules in zebrafish not only enables detection of adverse toxicity and off-target side effects in the early stages of pharmaceutical development¹⁷ , but has also led to the discovery of novel therapeutics currently undergoing clinical trials¹⁸ . However, most biologics cannot be absorbed from the water due to their high molecular weight or unfavorable physical and chemical properties, and delivery of biologics into animals often requires manual microinjection¹⁹, a process that is too slow and labor-intensive for HTS. Although automated microinjection systems have been developed for delivery of nucleic acids into the large yolk cells of zebrafish embryos immediately after fertilization²⁰, there is currently no high-throughput technology suitable for targeting specific organs of developed larvae and screening biologics in vivo, due to various technical challenges in different aspects of handling live larval zebrafish, including requirement of proper immobilization and orientation of larvae for micropipette to access different organs; difficulty to identify specific anatomic structures over transparent background; and lack of methods for parallel processing of multiple larvae. Thus, although zebrafish is an established model for study of human disease and also function of organs such as CNS, liver, kidney, and even blood brain barrier which are all relevant to delivery and processing of biologics, no study of biologics or delivery vehicle formulations have been reported using zebrafish.

### SUMMARY OF THE INVENTION

The automated system according to the invention for efficient delivery of biologics into target organs of zebrafish larvae for high-throughput in vivo screening includes a reservoir containing zebrafish larvae immersed in a hydrogel in its liquid state. A microfluidic component removes a droplet of the hydrogel having a single zebrafish larva contained therein and deposits the droplet on a surface for receiving an array of hydrogel droplets. Structure is provided for inducing the larvae to assume a dorsal or lateral orientation within the droplet. A temperature controller such as a thermoelectric device cools or heats the surface to solidify the hydrogel droplets thereby to immobilize the larvae and an optical arrangement identifies target organs in each larva using an image template-matching algorithm. A pressure driven microinjection needle injects biologics into the target organ of the zebrafish larva.

In a preferred embodiment, the structure for inducing a larva to assume a dorsal orientation is a motor causing the surface to vibrate. A substance for inducing a larva to assume a lateral position comprises introducing a mild anesthesia into the reservoir.

In another embodiment, are optical arrangement is adapted to examine phenotypic outcomes of the larvae. A suitable hydrogel is ultra-low gelling temperature agarose.

The microfluidic component preferably includes a multi-color, multi-angle, light-scattering and photo-detection system to discriminate individual larvae from debris and bubbles. It is also preferred that the microfluidic component deposits the droplets using a computer-controlled syringe pump in conjunction with a motorized x-y stage. It is also preferred that the surface be pre-patterned with an array of hydrophilic spots on a hydrophobic background.

A suitable mild anesthetic is tricane. Target organs include forebrain, midbrain, ventricles, eyes, heart and liver. Another preferred embodiment includes the optical arrangement having a computer running an algorithm to identify eyes and an anterior-posterior axis of a larva to serve as a reference coordinate. Suitable biologics include lipidoid-RNA complexes.

### BRIEF DESCRIPTION OF THE DRAWING

FIG. 1a is a schematic perspective illustration of the automated system disclosed herein.

FIG. 1b is a series of images of zebrafish larvae following automatic microinjection of FITC-coupled dextran molecules into different organs using the system of the invention.

### DESCRIPTION OF THE PREFERRED EMBODIMENT

We have developed an automated system for efficient delivery of biologics into target organs of zebrafish larvae for high-throughput in vivo screening. The system utilizes a microfluidic component under computer control to automatically distribute zebrafish larvae into an array of hydrogel droplets, each containing a single larva. While the hydrogel is still in a liquid state, vibrational stimulation or mild anesthesia is used to induce the larvae to assume either a dorsal or a lateral orientation. Subsequently, the substrate temperature is lowered causing the droplets to solidify and restrict all further motion. Next, the microinjection needle is automatically targeted to organs of interest using an image template-matching algorithm, and biologics are injected via a pressure driven system. Phenotypic outcomes, including in vivo distribution of biologics and gene expression, are then examined by optical imaging. Using this system, we screened a library of lipid-like compounds for their ability to facilitate the delivery and expression of oligonucleotides (protein-encoding RNAs) in the central nervous system (CNS) following injection into the cerebrospinal fluid (CSF) of the brain ventricles.

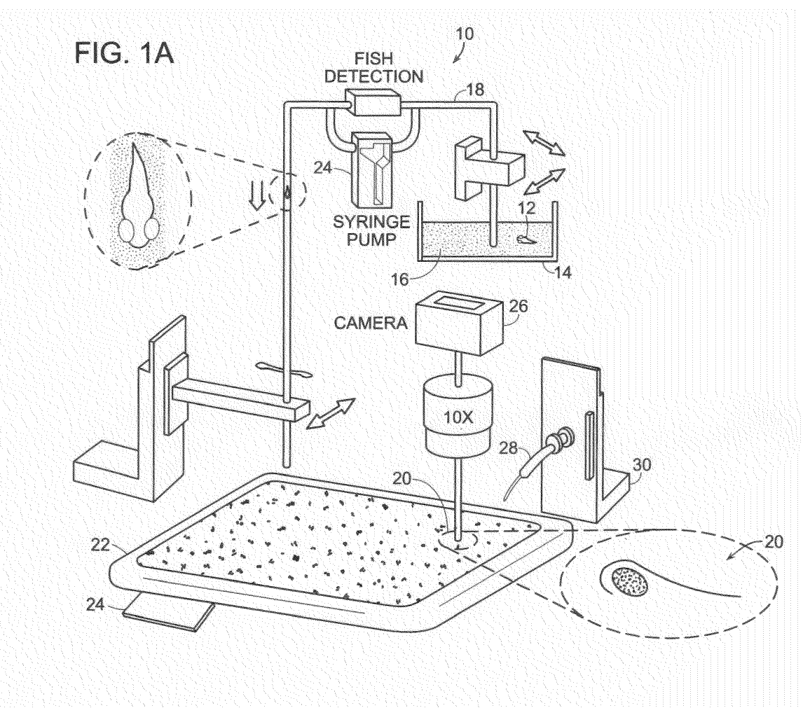

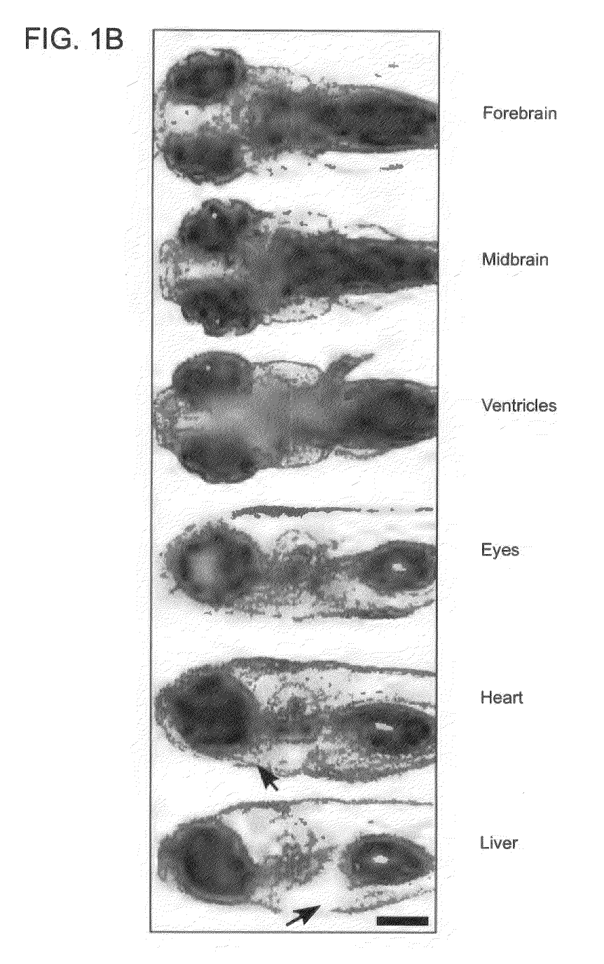

We developed an automated microinjection system 10 for high-throughput delivery of biologics to target tissues of zebrafish larvae at 4 days post fertilization, a stage at which all major organs have formed (FIG. 1a). Initially, zebrafish larvae 12 are placed in a heated reservoir 14 containing embryo medium 16 supplemental with 1% ultra-low gelling temperature agarose. The agarose-based hydrogel remains in the liquid phase at room temperature (25° C.) and solidifies when briefly lowered below 17° C. and increased back to 25° C. Brief exposure to this temperature range does not affect health of larvae²², as we also verify below in assessment of our overall procedure's effect on health. Zebrafish larva 12 are acquired from the reservoir 14 using a microfluidic component 18 we developed, which incorporates a multi-color, multi-angle, light-scattering and photo-detection system to discriminate individual larvae 12 from debris and bubbles and to guarantee successful acquisition of a single larva^{23,24}. Next, a hydrogel droplet 20 containing the larva 12 is deposited onto a flat plate 22 using a computer controlled syringe pump 24 and motorized X-Y stage. The plate 22 surface is pre-patterned with arrays of hydrophilic spots (96- or 48-well plate format) on a hydrophobic background, such that each hydrogel droplet remains confined within a precisely defined X-Y location in order to prevent mix-up with neighboring droplets. The use of hydrophilic spots surrounded by hydrophobic background allows generation of densely packed isolated droplets 20. We use droplet volumes large enough to avoid drying out, narrow enough to fit the array dimensions, and shallow enough to minimize the height of each hydrogel droplet to avoid optical distortion (25 μL for 96-spot arrays and 70 μL for 48-spot arrays).

The plate with arrays of larvae in liquid hydrogel droplets is transferred to a motorized X-Y stage with a thermoelectrically temperature-controlled substrate. To image and microinject to different organs of zebrafish, the larvae 12 are manipulated to adopt one of two major orientations. For injection into dorsal targets, larvae 12 within the hydrogel are agitated with several pulses of mechanical vibrations from a motor 24, which trigger a startle response that causes them to assume a dorsal-up orientation. For injection into lateral and ventral targets, larvae are anesthetized by addition of 0.2 mg/mL tricaine to the hydrogel solution in the reservoir 14, causing most to settle into a lateral orientation. After being properly oriented, the hydrogel droplets are solidified by cooling to 4° C. with a thermoelectric module associated with the plate 22, which results in effective immobilization of the larvae 12 within droplets 20. For larvae at 4 days post-fertilization (dpf), the success rates for dorsal and lateral orientation are 93±7% and 84±3%, respectively. With these methods, different organs within a larva, including forebrain, midbrain, ventricles, eyes, heart and liver, can be successfully targeted for microinjection (FIG. 1b).

Using an in-house developed image recognition program and a high-speed camera 26, the system automatically locates each larva 12 within a hydrogel-droplet 20, positions the larvae to the center of field of views, and zooms in with motorized z-focus. An algorithm identifies the eyes and the anterior-posterior axis of a larva, which can then be used as a reference coordinate to calculate the location of specific organs of interest. At the beginning of the microinjection process, a micropipette 28 is front-loaded with biologics from a multiwell plate and then lowered to approach the target tissue/organ surface. By comparing in real-time the image of the larva's exterior surface with the one from previous sampling point while the micropipette 28 approaches the target organ, the algorithms detect the distortion of the exterior surface by the needle prior to the needle's penetration into the larva. This allows our system to automatically not only identify the physical contact of the micropipette with the surface of the larva but also calculate its depth of penetration into the larva. Subsequently, a pressure-driven picoliter-precision injector 30 is triggered to deliver the biologics. The overall success rate of the automated microinjection into larval brain is 97% (n=150). While the successful injection rate for other organs could be lower due to different properties, such as size, location, and movement etc., the hardware and algorithms could be further tuned according to specific applications.

The average deviation of the automatically-targeted injection site from the desired site of injection (as determined by the user) is only 49±3 μm (distance±s.d., n=75 from 3 separate experiments), allowing highly precise targeted delivery into specific organs. After microinjection, a self-adhesive bottomless multiwell chamber (not shown) is attached onto the plate with arrays of larvae to isolate the hydrogel droplets from each other prior to a flushing process. The single-larva-containing hydrogel droplet in each well is then flushed with embryo medium to release the larvae from the droplets. It takes 20.0±0.9 seconds per larva on average to finish a complete cycle of loading, arraying, orientation, immobilization, target identification, microinjection, and recovery. This time can be further decreased to 13.1±0.5 seconds per larva by pipelining the steps of arraying and injection. This is considerably faster especially when compared to manual injection, which at least takes a trained technician several minutes^{19,25} to perform all the necessary procedures including anesthesia, immobilization, orientation of a single larva, and injection to the target organ. This is also exceptionally fast in practice, as one can screen thousands of delivery vehicle formulations/biologics in one week alone, which would otherwise take months to years if performed manually.

To evaluate whether the health of zebrafish larvae is affected by our system, we assessed 291 larvae using functional and morphological criteria (4 dpf) after passage through our system. Assessment of both survival and morphological abnormality (see methods) showed that our system caused no statistically significant adverse effects on zebrafish larvae with respect to controls.

The technology we have developed makes it possible for the first time to rapidly test numerous vehicle formulations for their ability to deliver RNA in vivo. The delivery scheme we used (i.e. injection of lipidoid-RNA complexes into CSF) is of direct clinical relevance, as lumbar intrathecal injection is anticipated to be a minimally invasive means for nonviral delivery to the CNS, and biologics delivered to the CSF has been shown to diffuse and distribute throughout extended regions of CNS in both rodents and humans^{29,36}. Our discovery of several vehicle formulations (C16-62, C16-120, C12-120) that are highly efficacious in rodent models without false positives suggest that zebrafish can be used as a model for high-throughput screening of biologics in vivo and, is more accurate than in vitro cell culture models in predicting outcomes to mammals. Interestingly, further analysis of our screening results also suggests certain structure-activity relationship, which can potentially be applied to design novel lipidoid delivery vehicles.

The reliability of the system depends on successful implementation of all operational procedures, including fish loading, immobilization/orientation, and microinjection. For example, we reported a success rate of ˜93% or ˜97% for dorsal orientation and ventricle injection, respectively. Given an almost 100% loading reliability, our system can perform brain injection with ˜90% reliability. It can potentially be used to automate and scale-up a variety of in vivo assays. For instance, zebrafish larvae have been shown to be a promising model for studying the blood-brain barrier and intravenous injection using our platform could be used to screen for vehicles that facilitate delivery of biologics from the circulatory system to the CNS. In addition, a number of disease models require precise delivery of cells to specific organs or body cavities. For example, human tumor cells have been injected into zebrafish to generate xenograft tumor models³⁷ and bacteria have been injected to model infection and pathogenesis³⁸. Using manual microinjection to generate sufficient numbers of animals for large-scale chemical screens would be too laborious. Our system can be used for rapid implantation of cells on a scale that is compatible with HTS of chemical libraries to identify anti-tumorigenic or anti-infectious drug leads.

Methods and Materials

Surface treatment for generating fish-arrays. Transparent hydrophobic polystyrene plates were plasma-treated with the protection of a PDMS mask containing arrays of holes (48- or 96-well format) to create circular hydrophilic spots over a hydrophobic background. The diameters of the 48- and 96-well spots are 8 mm and 5 mm, respectively.

Image processing for automated microinjection. A coordinate system is established using the centroids of the both eyes, the swim bladder, and the axis of the trunk as landmarks. The eyes and swim bladder are identified based on their contrast with other larval surface features using a threshold-based segmentation algorithm. An image of the larva embedded in agarose is first captured by a high-speed CCD camera (GX-1050, Prosilica) through a Nikon AZ-100 Multizoom microscope and then converted to a binary image using a threshold, where the threshold value is determined via statistical analysis of the overall illumination level of the image. Next, the objects in the binary image are filtered to eliminate smaller high-contrast objects such as melanocytes, leaving only the eyes and swim bladder. The filtering is performed by removing pixel-connected objects composed of pixels less than a threshold value. The threshold size is automatically adjusted to obtain only 3 objects from the images. Since the eyes are located closer to each other than they are to the swim bladder, the two objects with the least distance between their centroids are designated as eyes and the remaining object is recognized as the swim bladder. The anterior-posterior axis can be determined either by using curve-fitting along the centroids of eyes and swim bladder or by rotation image-correlation with a reference image of larva.

Automated injection is then performed by diagonally lowering the injection micropipette (Micromanipulator: Patchman NP2, Eppendorf; Injector: Xenoworks, Sutter Instrument) to approach the target while monitoring the difference between real-time images and the pre-injection images to detect the contact and penetration of the micropipette tip. Specifically, after the micropipette tip contacts the exterior of the larva, but before it actually penetrates any tissue, the difference between the real-time images and the pre-injection images increases dramatically as the tissue is pressed by the tip and deforms. Following the penetration of the micropipette into the tissue, the image difference decreases as the tissue deformation relaxes. After penetration is detected, a 1 nL volume is injected by triggering a pressure drive picoliter microinjector (Sutter Instrument). Following injection, the micropipette is retracted to the home position. The automation control of microinjector and data readout is through NIDAQ cards (NI9422; NI USB-6211). Software is developed on Matlab.

Health assessment of larvae processed by the system. For health assessment and all subsequent experiments, the syringe pump was operated at aspiration rates of 330 μL/s. 4 dpf larvae were loaded from a reservoir, deposited onto the surface-treated plate, microinjected with 1 nL of PBS, and recovered for assessment by briefly flushing the surface of each hydrogel droplet with low-pressure stream of embryo medium. In total, 291 larvae were processed and compared with a control group of 187 larvae from the same clutch. Health assessment was based on both functional and morphological criteria. Functional criteria included visual confirmation of normal heartbeat and reflex response to touch stimuli. Morphological criteria included spine bending (i.e. lordosis, kyphosis, and scoliosis) and craniofacial abnormalities³⁹. Larvae were assessed immediately after recovery from the hydrogel droplets and again every 24 hours over the course of the next 4 days.

More details of the invention and of experiments conducted therewith may be found in “Organ-targeted high-throughput in vivo biologics screen identifies materials for RNA delivery” by Chang et al. Integrative Biology, Volume 6 Number 10, 926 (Aug. 5, 2014), the contents of which are incorporated herein by reference and constituting the work of the present inventors. The other references listed herein are also incorporated by reference in their entirety.

### REFERENCES

1., J. C. Burnett and J. J. Rossi, Chemistry & biology, 2012, 19, 60-71 .

2. A. S. Harms, C. J. Barnum, K. A. Ruhn, S. Varghese, I. Trevino, A. Blesch and M. G. Tansey, Molecular therapy: the journal of the American Society of Gene Therapy, 2011, 19, 46-52.

3. W. Stohl and D. M. Hilbert, Nature biotechnology, 2012, 30, 69-77.

4. S. U. Kim and J. de Vellis, Journal of neuroscience research, 2009, 87, 2183-2200.

5. F. Alexis, E. M. Pridgen, R. Langer and O. C. Farokhzad, Handbook of experimental pharmacology, 2010, 55-86.

6. A. Akinc, A. Zumbuehl, M. Goldberg, E. S. Leshchiner, V. Busini, N. Hossain, S. A. Bacallado, D. N. Nguyen, J. Fuller, R. Alvarez, A. Borodovsky, T. Borland, R. Constien, A. de Fougerolles, J. R. Dorkin, K. Narayanannair Jayaprakash, M. Jayaraman, M. John, V. Koteliansky, M. Manoharan, L. Nechev, J. Qin, T. Racie, D. Raitcheva, K. G. Rajeev, D. W. Sah, J. Soutschek, I. Toudjarska, H. P. Vomlocher, T. S. Zimmermann, R. Langer and D. G. Anderson, Nature biotechnology, 2008, 26, 561-569.

7. A. D. Judge, V. Sood, J. R. Shaw, D. Fang, K. McClintock and I. MacLachlan, Nature biotechnology, 2005, 23, 457-462.

8. D. B. Rozema, D. L. Lewis, D. H. Wakefield, S. C. Wong, J. J. Klein, P. L. Roesch, S. L. Bertin, T. W. Reppen, Q. Chu, A. V. Blokhin, J. E. Hagstrom and J. A. Wolff, Proceedings of the National Academy of Sciences of the United States of America, 2007, 104, 12982-12987.

9. T. S. Zimmermann, A. C. Lee, A. Akinc, B. Bramlage, D. Bumcrot, M. N. Fedoruk, J. Harborth, J. A. Heyes, L. B. Jeffs, M. John, A. D. Judge, K. Lam, K. McClintock, L. V. Nechev, L. R. Palmer, T. Racie, I. Rohl, S. Seiffert, S. Shanmugam, V. Sood, J. Soutschek, I. Toudjarska, A. J. Wheat, E. Yaworski, W. Zedalis, V. Koteliansky, M. Manoharan, H. P. Vomlocher and I. MacLachlan, Nature, 2006, 441, 111-114.

10. C. Falschlehner, S. Steinbrink, G. Erdmann and M. Boutros, Biotechnology journal, 2010, 5, 368-376.

11. K. T. Love, K. P. Mahon, C. G. Levins, K. A. Whitehead, W. Querbes, J. R. Dorkin, J. Qin, W. Cantley, L. L. Qin, T. Racie, M. Frank-Kamenetsky, K. N. Yip, R. Alvarez, D. W. Sah, A. de Fougerolles, K. Fitzgerald, V. Koteliansky, A. Akinc, R. Langer and D. G. Anderson, Proceedings of the National Academy of Sciences of the United States of America, 2010, 107, 1864-1869.

12. X. Yang, N. Li and D. G. Gorenstein, Expert opinion on drug discovery, 2011, 6, 75-87.

13. P. Shi, M. A. Scott, B. Ghosh, D. Wan, Z. Wissner-Gross, R. Mazitschek, S. J. Haggarty and M. F. Yanik, Nat Commun, 2011, 2, 510.

14. G. J. Lieschke and P. D. Currie, Nature reviews. Genetics, 2007, 8, 353-367.

15. C. Parng, W. L. Seng, C. Semino and P. McGrath, Assay and drug development technologies, 2002, 1, 41-48.

16. L. I. Zon and R. T. Peterson, Nature reviews. Drug discovery, 2005, 4, 35-44.

17. P. M. Eimon and A. L. Rubinstein, Expert opinion on drug metabolism & toxicology, 2009, 5, 393-401.

18. T. E. North, W. Goessling, C. R. Walkley, C. Lengerke, K. R. Kopani, A. M. Lord, G. J. Weber, T. V. Bowman, I. H. Jang, T. Grosser, G. A. Fitzgerald, G. Q. Daley, S. H. Orkin and L. I. Zon, Nature, 2007, 447, 1007-1011.

19. J. H. Gutzman and H. Sive, Journal of visualized experiments: JoVE, 2009.

20. W. Wang, X. Liu, D. Gelinas, B. Ciruna and Y. Sun, PloS one, 2007, 2, e862.

21. J. G. Nutt, K. J. Burchiel, C. L. Cornella, J. Jankovic, A. E. Lang, E. R. Laws, Jr., A. M. Lozano, R. D. Penn, R. K. Simpson, Jr., M. Stacy and G. F. Wooten. Neurology, 2003, 60, 69-73.

22. Y. Long, G. Song, J. Yan, X. He, Q. Li and Z. Cui, BMC Genomics, 2013, 14, 612.

23. T. Y. Chang, C. Pardo-Martin, A. Allalou, C. Wahlby and M. F. Yanik, Lab on a chip, 2012, 12, 711-716.

24. C. Pardo-Martin, T. Y. Chang, B. K. Koo, C. L. Gilleland, S. C. Wasserman and M. F. Yanik, Nature methods, 2010, 7, 634-636.

25. J. L. Cocchiaro and J. F. Rawls, Journal of visualized experiments: JoVE, 2013, e4434.

26. G. F. Jirikowski, P. P. Sanna, D. Maciejewski-Lenoir and F. E. Bloom, Science, 1992, 255, 996-998.

27. M. S. Kormann, G. Hasenpusch, M. K. Aneja, G. Nica, A. W. Flemmer, S. Herber-Jonat, M. Huppmann, L. E. Mays, M. Illenyi, A. Schams, M. Griese, I. Bittmann, R. Handgretinger, D. Hartl, J. Rosenecker and C. Rudolph, Nature biotechnology, 2011, 29, 154-157.

28. J. M. Vargason, G. Szittya, J. Burgyan and T. M. Hall, Cell, 2003, 115, 799-811.

29. P. Leone, C. G. Janson, L. Bilaniuk, Z. Wang, F. Sorgi, L. Huang, R. Matalon, R. Kaul, Z. Zeng, A. Freese, S. W. McPhee, E. Mee and M. J. During, Ann Neurol, 2000, 48, 27-38.

30. Y. J. Cao, T. Shibata and N. G. Rainov, Gene therapy, 2002, 9, 415-419.

31. R. Blum, C. Heinrich, R. Sanchez, A. Lepier, E. D. Gundelfinger, B. Berninger and M. Gotz, Cereb Cortex, 2011, 21, 413-424.

32. C. Kizil, N. Kyritsis, S. Dudezig, V. Kroehne, D. Freudenreich, J. Kaslin and M. Brand, Dev Cell, 2012, 23, 1230-1237.

33. S. Robel, B. Berninger and M. Gotz, Nat Rev Neurosci, 2011, 12, 88-104.

34. M. Angel and M. F. Yanik, PloS one, 2010, 5, e11756.

35. K. Kariko, H. Muramatsu, F. A. Welsh, J. Ludwig, H. Kato, S. Akira and D. Weissman, Molecular therapy: the journal of the American Society of Gene Therapy, 2008, 16, 1833-1840.

36. D. M. Anderson, L. L. Hall, A. R. Ayyalapu, V. R. Irion, M. H. Nantz and J. G. Hecker, Hum Gene Ther, 2003, 14, 191-202.

37. A. M. Taylor and L. I. Zon, Zebrafish, 2009, 6, 339-346.

38. K. Takaki, C. L. Cosma, M. A. Troll and L. Ramakrishnan, Cell Rep, 2012, 2, 175-184.

39. S. R. Blechinger, J. T. Warren, Jr., J. Y. Kuwada and P. H. Krone, Environ Health Perspect, 2002, 110, 1041-1046.

What is claimed is:

1. Automated system for efficient delivery of biologics into target organs of zebrafish larvae for high-throughput in vivo screening comprising:
a reservoir containing zebrafish larvae immersed in a temperature sensitive hydrogel in its liquid state;
a microfluidic component for removing a droplet of the hydrogel having a single zebrafish larva contained therein and depositing the droplet on a surface for receiving an array of hydrogel droplets;
means for inducing the larva to assume a dorsal or lateral orientation within the droplet;
a controller for controlling temperature of the surface to solidify the temperature-sensitive hydrogel droplets thereby to immobilize the larvae;
an optical arrangement to identify target organs in each larva using an image template-matching algorithm; and
a pressure driven microinjection needle for injecting biologics into the target organ of the zebrafish larva.

2. The system of claim 1 wherein the means for inducing the larvae to assume a dorsal orientation is a motor causing the surface to vibrate.

3. The system of claim 1 wherein the means for inducing the larva to assume a lateral position comprises introducing a mild anesthesia into the reservoir.

4. The system of claim 1 wherein the optical arrangement is adapted to examine phenotypic outcomes of the larvae.

5. The system of claim 1 wherein the hydrogel is ultra-low gelling temperature agarose.

6. The system of claim 1 wherein the microfluidic component includes a multi-color, multi-angle, light-scattering and photo-detection system to discriminate individual larvae from debris and bubbles.

7. The system of claim 1 wherein the microfluidic component deposits the droplet using a computer-controlled syringe pump in conjunction with a motorized x-y stage.

8. The system of claim 1 wherein the surface is pre-patterned with an array of hydrophilic spots on a hydrophobic background.

9. The system of claim 3 wherein the mild anesthesia is tricane.

10. The system of claim 1 wherein the target organ includes forebrain, midbrain, ventricles, eyes, heart and liver.

11. The system of claim 1 wherein the optical arrangement includes a computer running an algorithm to identify eyes and an anterior-posterior axis of a larva to serve as a reference coordinate.

12. The system of claim 1 wherein the biologics include lipidoid-RNA complexes.
